(19) United States
(12) Patent Application Publication
(10) Pub. No.: US 2026/0162822 A1
(43) Pub. Date: Jun. 11, 2026

(54) Mammography Deep Learning Model

(71) Applicants: Agfa Healthcare NV, Mortsel (BE); VRVIS Zentrum für Virtual Reality und Visualisierung Forschungs-GmbH, Vienna (AT)

(72) Inventors: Maria Wimmer, Mortsel (BE); David Major, Mortsel (BE); Dimitrios Lenis, Mortsel (BE); Astrid Berg, Mortsel (BE); Katja Buehler, Mortsel (BE)

(73) Assignees: Agfa Healthcare NV, Mortsel (BE); VRVIS Zentrum für Virtual Reality und Visualisierung Forschungs-GmbH, Vienna (AT)

(21) Appl. No.: 18/706,948

(22) PCT Filed: Oct. 19, 2022
(86) PCT No.: PCT/EP2022/079005

§ 371 Date: May 2, 2024

(30) Foreign Application Priority Data
Nov. 5, 2021 (EP) 21206588.2

(51) Int. Cl. G16H 50/20; G06N 20/00; G16H 30/40

(52) U.S. Cl. CPC G16H 50/20; G06N 20/00; G16H 30/40

(57) ABSTRACT

A computer-implemented method for developing a mammography deep learning model wherein a set of task-specific mammography deep learning models is developed, each trained for performing a different task on a mammography dataset and each generating one or more feature vectors and wherein the task-specific models are combined to a patient model to obtain a patient prediction by fusing said feature vectors.

### TECHNICAL FIELD

The present invention relates to a computer-implemented method for developing a mammography deep learning model.

### Background of the Invention

### SUMMARY OF INVENTION

Breast cancer is the most common cancer type in women and also the leading cause of death by cancer in women worldwide. Fortunately, the mortality rate declined in recent years, one reason being the higher rate of early diagnosis due to the establishment of screening programs.

Important cancer risk factors, such as breast density, can be detected and monitored early with such programs.

Due to the increasing amount of imaging data, machine learning, especially deep learning algorithms are being developed to process mammography data automatically. Such models perform, for example, localization and classification of lesions, breast density classification, or cancer risk prediction. These automated methods can be used for accelerating reading workflows, or ideally, to support radiologists in their image interpretation and diagnosis. Several recent studies further report higher accuracies when combining AI algorithms with the assessment of a single radiologist, or improved performance of radiologists when aided by an AI system.

Besides the obtained performance gains, the assistance of radiologists as well as human-computer collaboration are becoming increasingly important aspects and challenges for future application in clinical practice. To increase trust in AI support tools, not only the interpretability of black box models is being intensively studied but also the potential of providing intermediate model results that are linked to radiological features. Recent user studies in cancer screening and diagnosis showed that clinicians profited more from models that provide detailed results compared to solutions delivering solely a benign/malignant assessment.

A standard mammography study comprises four X-ray images that correspond to two different imaging views from each breast: L-CC, R-CC, L-MLO, and R-MLO. Thereby, CC corresponds to the craniocaudal (CC) view, MLO to the mediolateral oblique (MLO) view, and L and R indicate the left or right breast, respectively. Radiologists analyze each view in detail and compare them to obtain a comprehensive view of a patient and render a diagnostic decision. Suspicious lesions, for example, can be visible in one view of a breast but may be obscured in the other view. Therefore, a thorough analysis is necessary. Various deep learning based methods have been presented in the past years that analyze single- or multiple-view images at a time. However, this is strongly dependent on their task and related clinical question.

Many methods have been described in the literature, for example for breast density scoring, lesion localization and classification, malignancy scoring and feature or information fusion.

While many recent works directly classify regions of interest (ROIs) or view images with, e.g., Convolutional Neural Networks (CNNs), a significant part utilizes some form of fusion when processing mammography data. The reasons are manifold: fusion is performed to (i) incorporate different aspects at different levels (ROI, image, patient), (ii) thus, increase robustness and performance of classification models, and (iii) increase explainability and interpretability of model predictions.

However, methods that perform a fusion of features within or across images mostly do not provide intermediate results (e.g., assessment of suspicious regions) but only final classification results. On the other hand, methods that fuse predictions across one or more ROIs or mammograms build upon models that predict the same scores or perform standard model ensembling strategies.

It is an aspect of the present invention to provide an enhanced strategy.

The above-mentioned aspects are realized by a method having the specific method steps set out in claim 1. Specific features for preferred embodiments of the invention are set out in the dependent claims.

Further advantages and embodiments of the present invention will become apparent from the following description and drawings.

The present invention focusses on information fusion for mammography from another perspective by focusing on the fusion of features and predictions from individual, task-specific models to obtain a comprehensive assessment on patient level.

In the context of the present invention, a model refers to a deep neural network that consists of one or more input layers, a sequence of non-linear transformations of the inputs and an output layer.

To the above-described end a pipeline approach is proposed that comprises
the development of three task-specific models, namely (i) a breast density classification model, (ii) a lesion localization model, (iii) and a findings classifier, as a basis for fusion, and
the investigation of two fusion strategies: (i) the fusion of high-dimensional, task-specific CNN features with a multi-input embedding CNN, and (ii) prediction score fusion of model predictions with multilayer perceptrons (MLPs).

By building upon task-specific features and decisions, hybrid patient meta-models are obtained which access these intermediate results in their prediction.

Due to the two-stage nature of the method, not only a global score on the patient level is reported but also sub-results are made accessible to the clinician, these sub-results reflecting radiological features.

Both fusion approaches are trained for two different classification targets, which will be referred to as patient predictions (i.e., prediction of the respective model).

The following is predicted: (1) the presence of any lesion (lesion prediction) and (2) whether the patient has any malignant lesion (malignancy prediction).

This is achieved by utilizing lightweight architectures like MobileNets for image classification-related tasks. The full pipeline was trained and evaluated on the well-known and publicly available DDSM and CBIS-DDSM datasets. It can be shown that the task fusion strategy of the present invention improves patient-level classification over standard model ensembling.

### BRIEF DESCRIPTION OF DRAWINGS

FIG. 1 is a density view model Du for view v E {L-CC, L-MLO, R-CC, R-MLO},

FIG. 2 represents Density patient model D,

FIG. 3 represents Finding model F,

FIG. 4 represents Localization model L,

FIG. 5 represents Patient meta-model P_{feat}.

### DETAILED DESCRIPTION OF THE INVENTION

A set of mammography images

$${I}_{i} = \{ {I}_{i}^{v} \}$$

for Patient i and mammography image view v∈{L-CC, L-MLO, R-CC, R-MLO} are defined. This set Iᵢ will be referred to as exam or case of patient i.

Two publicly available mammography databases were utilized for these experiments: the Digital Database for Screening Mammography (DDSM) and its curated version CBIS-DDSM.

1) DDSM and CBIS-DDSM Dataset: The original DDSM dataset comprises 2620 mammography screening exams Iᵢ, collected from four different sites acquired with four different scanners. The data is grouped in four categories:
normal (695 cases): normal exams with no suspicious abnormalities and proven normal exams four years later
benign without callback (141 cases): cases with benign abnormality but without need for callback
benign (870 cases): including suspicious findings which were identified as benign findings after callback
cancer (914 cases): cancer was proven via histology

An expert radiologist labeled the breast density per patient and provided pixel-level annotation for abnormalities. Each abnormality is described following the BI-RADS standard, including lesion type (mass or calcification) and further details like shape, lesion margin, and calcification type.

The CBIS-DDSM dataset was published at The Cancer Imaging Archive as curated version of the original DDSM set, whereby only images showing one or more lesions have been transferred. Annotated masses were re-checked by a radiologist, and pixel-wise annotations have been refined with an automated segmentation algorithm. However, annotations of calcifications remained unchanged. The authors also provided a predefined split into train and test sets to ensure comparability between methods evaluated on this dataset. Overall, the CBIS-DDSM dataset comprises 3568 annotated lesions (1696 masses, 1872 calcifications) in a total of 3032 mammography view images.

2) Data Harmonization and Preparation: While providing enhanced annotation quality, the CBIS-DDSM dataset has two shortcomings: first, the absence of normal images without lesions, and second, the lack of full patient mammography exams including all four views. To utilize both resources without losing their individual benefits, we prepare the data as follows:

First we preprocess the DDSM set in the same way as it was done for the CBIS-DDSM data, including optical densitynormalization and remapping the data to the full 16-bit range.

Next, we match, i.e., compare the CBIS-DDSM images to the preprocessed DDSM data to identify corresponding cases and obtain a total of 2590 full mammography exams. We assign the malignancy status of a lesion according to the curated annotation from CBIS-DDSM, whereby “benign without callback” will be treated as a benign case.

Finally, we identify potential ambiguous cases which have been originally in the cancer, benign, or benign without callback subset in DDSM but have not been transferred to CBIS-DDSM. Since the status of the lesions for these 329 cases remains unclear, we exclude them. Further, we exclude seven additional exams, which are either incomplete, i.e., not all four views are present, or appeared with different imaging data and annotations in different subsets of DDSM and CBIS-DDSM. This leads to our final set comprising 2254 cases.

3) Train, Validation, Test Split: We split the date set into train, validation and test data on case-level and, thus, ensure that images from one case are not distributed across different sets. We preserve the train/test split of the data provided with the CBIS-DDSM set. The remaining normal cases are randomly distributed in the same ratio (˜80% training images) to the train/test set in a way that the distribution of breast density is similar in the three sets. From the obtained train set, we randomly select ˜12% of cases for the validation set in a way that the ratio of different breast density classes, lesion types, and pathology is similar across the three sets. Overall, the train, validation, and test set comprise 1511, 290, and 453 cases, respectively. Out of 2254 cases, 174 contain more than one lesion, with the maximum number of lesions per case being 24.

### Task-Specific Mammography Models:

The first stage in our pipeline is the development of a set M of three resource-efficient, task-specific models M={D, L, F}, which are the base for our patient model P:
D performs breast density classification,
L delivers bounding boxes around localized lesions and their respective class label, and
F predicts the presence/absence of lesions in an image.

Breast Density Model (D): Radiologists include all four view images Iᵢ in the assessment of a patient's breast density. Recent deep learning based density classification models follow this standard and utilize all views as input, whereas the usage of only one view has also been studied. We propose a two-stage approach where we employ both ideas in the design of density model D to increase robustness and classification performance.

We build a view model D^{v} first, which uses any single mammography image

$${I}_{i}^{v}$$

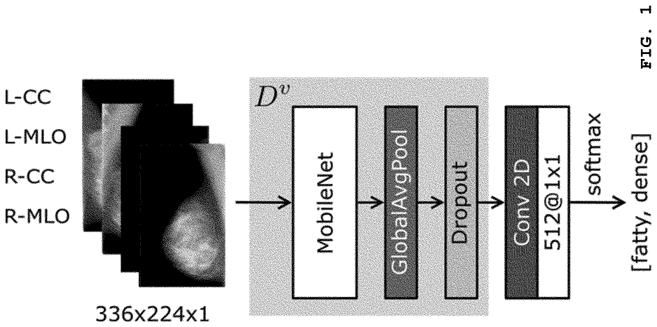

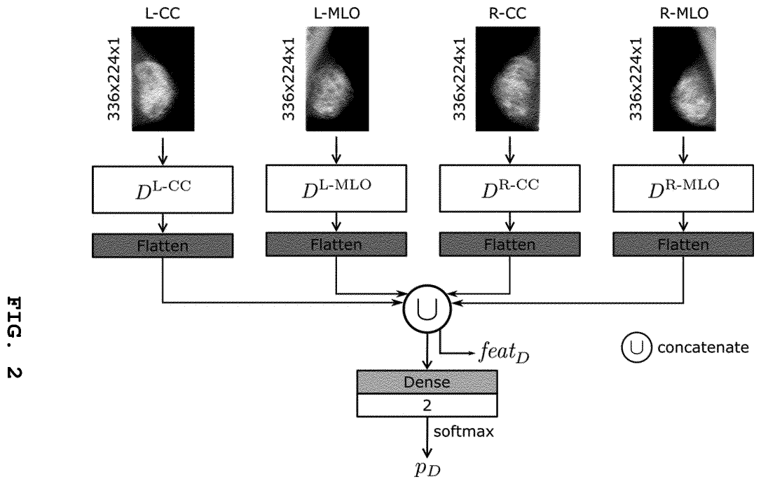

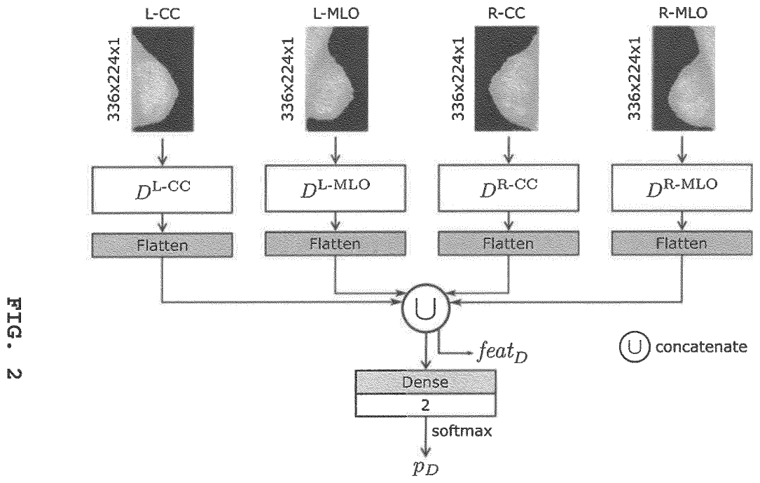

as input to predict the density super-class, i.e., fatty or dense. The model is built upon a MobileNet classifier with global average pooling, followed by a 1×1 convolution layer (see FIG. 1). Our final model D takes the four standard mammography views Iᵢ as input where each image is passed to a separate branch (see FIG. 2).

Each view branch consists of a density view model Dᵥ, whereby the dropout rate is increased from 0.001 in model Dᵥ to 0.5 in D. After the following flattening operation, the 1D feature vectors are concatenated, and a final dense layer predicts the density superclass. The obtained density score p_{D} at patient-level depicts the score corresponding to the “dense” class.

Findings model (F): The objective of this model is to classify any single-view image

$${I}_{i}^{v}$$

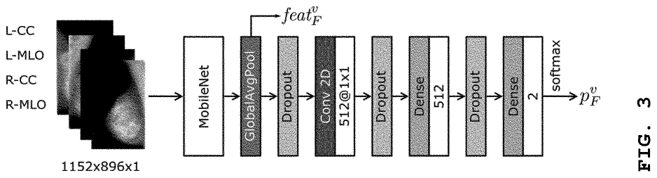

into “normal” or “image containing any findings”, i.e., lesions. Such a model could be, for example, integrated in a reporting system, in which images with lesions are examined first by a medical expert. We apply MobileNet in this context. FIG. 3 illustrates our findings model F with a MobileNet feature extractor and a modified classifier on top. Adding an additional dense and dropout layer increased the classification accuracy and the generalization capability of the model.

Additionally we use an increased dropout rate of 0.5 to stronger regularize the network. The output for each view image

$${I}_{i}^{v}$$

is the score

$${p}_{F}^{v}$$

which determines whether there is any lesion in

$${I}_{i}^{v} .$$

Localization Model (L): Similar to radiologists, we aim to detect the exact location of lesions within an image

$${I}_{i}^{v}$$

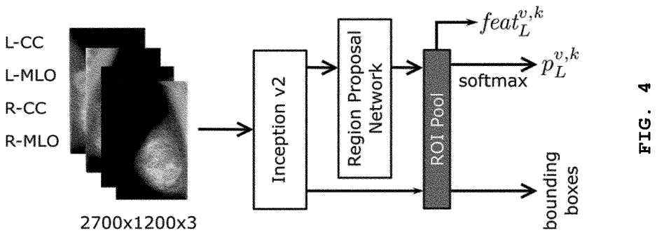

and classify them into their correct type and malignancy status. The localization and characterization of lesions are important tasks, as they can be risk factors or already indicators of cancer. Therefore, we develop model L to localize lesions and classify them in either “benign calcification”, “malignant calcification”, “benign mass”, or “malignant mass”. We utilize the well-known Faster R-CNN architecture. InceptionV2 serves as feature extractor, which was already successfully applied in the context of mammography lesion localization. FIG. 4 illustrates the architecture. Our localization model L classifies localized lesions into four types (benign calcification, malignant calcification, benign mass, and malignant mass) and assigns k∈[0, n] scores

$${p}_{L}^{v , k} ,$$

depending on the number of detected lesions that are found in

$${I}_{i}^{v} .$$

### Patient Meta-Model (P)

The aim of the hybrid patient meta-model P is to efficiently combine the task-specific building blocks M to obtain a comprehensive patient-level assessment while preserving the individual model predictions that are related to radiological features and risk factors. We consider two different patient predictions:
Lesion prediction: whether the patient has any lesion, regardless of pathology,
Malignancy prediction: whether the patient is malignant, i.e., has any malignant lesion.

The fusion of different models can be performed at various stages, whereby, again, our goal is to develop resource-efficient variants. For this, we compare the fusion of prediction scores as well as the fusion of features from the individual models.

1) Fusion of predictions (P_{score}): The three task models deliver different prediction scores pₘ∈[0,1], m∈M at various levels, i.e., patient level, image level, ROI level.

We concatenate these predictions of the models introduced higher in the section about Task-specific mammography models to form the vector wₚ, formally

$${w}_{p} = {p}_{D} ⋃ {p}_{F}^{v} ⋃ {p}_{L}^{v , n}$$

where n is the number of considered detections per view. In case of no detected lesions by model L or less lesions than specified by n are found, a probability of 0 is assigned, indicating that no (additional) lesions have been localized. For the malignancy prediction, only scores

$${p}_{L}^{j , n}$$

corresponding to malignant masses and calcifications are considered in the combined score vector wₚ. In case no malignant lesions or less malignant lesions than specified by n are found, a value of 0 is assigned.

2) Fusion of features (P_{feat}): Apart from the fusion of prediction scores pₘ>we also propose the fusion of feature vectors featₘ, m∈M from the three different models.

We extract features at the following stage in the networks:

feat_{D} is the 4096-dimensional, flattened, concatenated view representations after average pooling (see FIG. 2)

$${feat}_{F}^{v}$$

is the 1024-dimensional representation for view image

$${I}_{i}^{v} ,$$

obtained after global average pooling (see FIG. 3)

$${feat}_{L}^{v , k}$$

is the 1024-dimensional representation for detection k in

$${I}_{i}^{v}$$

### (see FIG. 4)

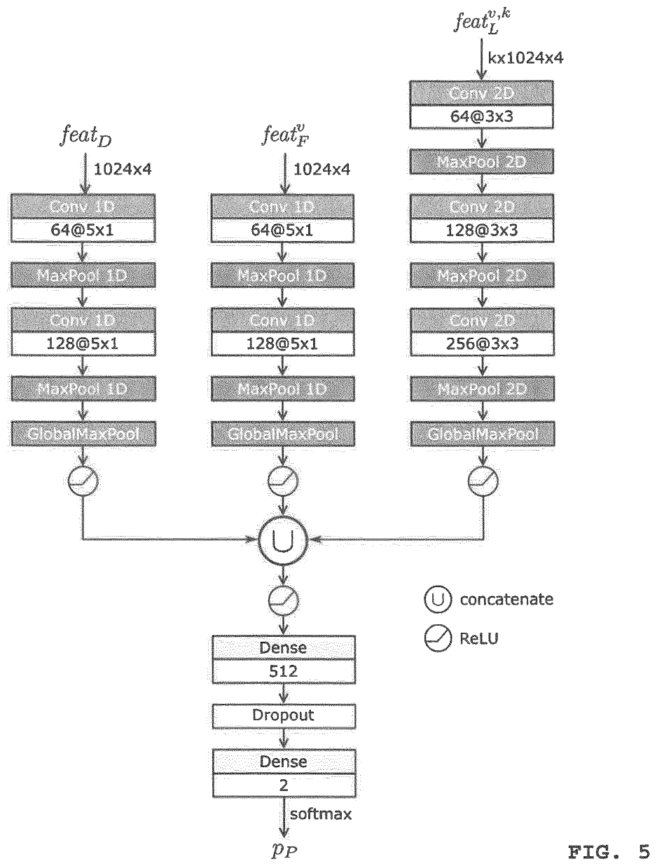

We propose an embedding network that takes the extracted, high-dimensional feature representations featₘ as input in separate branches (see FIG. 5). Each channel corresponds to the respective features of a view image

$${I}_{i}^{v} .$$

The density and findings branches consist of two convolution blocks, followed by pooling operations. The localization feature branch utilizes an additional convolution and pooling block for better feature learning. Before and after concatenation of all feature representations, we perform ReLU (rectified linear unit) activations. The final classification part of the network consists of two dense layers with an intermediate dropout layer (dropout rate of 0.1) followed by a final softmax activation.

Again, we vary the number of lesions considered per view n∈{1, 2, 3, 4, 5}. In case no lesions are detected with model L, or less lesions than specified by n, background features are pooled from the feature map and used as input. For the malignancy prediction, only features

$${feat}_{L}^{j , n}$$

corresponding to malignant masses and calcifications according to the localization model L are considered for feature fusion. In case of no malignant lesions or less than specified by n, again background features are considered as model input.

1. A computer-implemented method for developing a mammography deep learning model comprising the steps of:
developing a set of task-specific mammography deep learning models each trained for performing a different task on a mammography dataset and each generating one or more feature vectors,
said set of task-specific models at least comprising one model which classifies the breast density from mammography view images, one model which delivers bounding boxes around localized lesions and their respective class label from a mammography view image and one model that predicts the presence or absence of a lesion from a mammography view image,
said feature vectors corresponding to high-dimensional representations of intermediate layers of said task-specific models, and
combining said task-specific models to obtain different types of patient predictions by fusing said feature vectors, said patient predictions describing (1) if a patient has any malignant lesion and (2) if the patient has any lesion regardless of the pathology of the lesion.

2. The method according to claim 1, wherein said task-specific feature vectors are fused by means of a deep learning model, said model comprising multiple input branches, each of these branches being specific to the feature vectors for a different task and each branch transforming said feature vectors by a series of convolutional and pooling layers, and then fusing them by concatenation, whereby fused feature vectors are being transformed by a series of dense layers before obtaining said patient prediction.
